(19) United States
(12) Patent Application Publication
(10) Pub. No.: US 2022/0296249 A1
(43) Pub. Date: Sep. 22, 2022

(54) ENDOVASCULAR COIL DEVICE FOR EMBOLIZATION OF BLOOD VESSELS

(71) Applicants: Rhode Island Hospital, Providence, RI (US); Brown University, Providence, RI (US)

(72) Inventors: Aaron W. P. MAXWELL, Providence, RI (US); Edward G. WALSH, Danielson, CT (US)

(21) Appl. No.: 17/695,209

(22) Filed: Mar. 15, 2022

Related U.S. Application Data
(60) Provisional application No. 63/161,750, filed on Mar. 16, 2021.

(51) Int. Cl. A61B 17/12; A61B 17/00

(52) U.S. Cl. CPC A61B 17/12113; A61B 17/1214; A61B 17/00234; A61B 2017/00238; A61B 2017/00292; A61B 2017/00871; A61B 2017/00004

(57) ABSTRACT

An endovascular coil includes a radiolucent permanent shape memory or shape-retaining polymer core that retains its coil shape with passage through a catheter, and a temporary degradable radiopaque coating surrounding the permanent shape memory or shape-retaining polymer core.

### CROSS REFERENCE TO RELATED APPLICATIONS

This application claims benefit from U.S. Provisional Patent Application Ser. No. 63/161,750, filed Mar. 16, 2021, which is incorporated by reference in its entirety.

### BACKGROUND OF THE INVENTION

The present invention relates generally to minimally-invasive image-guided procedures, and more particularly to an endovascular coil device for embolization of blood vessels.

In general, in vascular disorders such as aneurysm, arteriovenous malformation and arteriovenous fistula, and in the embolization treatment of internal bleeding related to traumatic injury or other causes, and in an embolization treatment (or therapy) of the nutrient artery to tumor or the like, an endovascular treatment is performed using an embolization material. That is, a tip of a catheter is directed to the vicinity of the treatment site and the embolization material is injected into the treatment site through the tip. Alternatively, a guide wire containing the embolization material in the tip is inserted into the treatment site through this catheter, and thus the embolization material portion is separated and is retained at the treatment site. Blood flow to the treatment site is blocked by the embolization material per se and thrombus formed on the embolization material, and the treatment is performed. Traditionally, a fine coil made of a platinum alloy is suitably used as such the embolization material.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features an endovascular coil including a radiolucent permanent shape memory or shape-retaining polymer core that retains its coil shape with passage through a catheter, and a temporary degradable radiopaque coating surrounding the permanent shape memory or shape-retaining polymer core.

In another aspect, the invention features an endovascular coil device including a catheter, and an endovascular coil, the endovascular coil including a radiolucent permanent shape memory or shape-retaining polymer core, and a temporary degradable radiopaque coating surrounding the permanent shape memory or shape-retaining polymer core.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 illustrates exemplary coils.

FIG. 2 illustrates a cross section of an exemplary coil.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

Health care providers use endovascular coiling, also called endovascular embolization, to block blood flow into an aneurysm. Aneurysms can be located in the brain or other parts of the body. An aneurysm is a weakened area in the wall of an artery. If an aneurysm ruptures, it can cause life-threatening bleeding and damage to the brain or other parts of the body where the aneurysm is location, as well as death. Preventing blood flow into an aneurysm helps to keep it from rupturing. Endovascular coiling is also used to treat many other causes of internal bleeding, including but not limited to arteriovenous malformations, arteriovenous fistulae, and blood vessel injuries as may be caused by trauma, for example, where the blood vessel is damaged. Moreover, endovascular coiling is used to block blood flow from one or more arteries that provide nutrition and oxygen to cancerous and noncancerous tumors.

More particularly, for endovascular coiling, health-care providers typically use a catheter, a long, thin tube inserted into an artery in the wrist or groin. The catheter is advanced into the affected artery in the body where the coil is deployed. X-ray imaging helps guide the catheter into the artery. The coils are typically made of soft platinum metal, and are shaped like a spring. These coils are very small and thin, ranging in size from about twice the width of a human hair to less than one hair's width.

Thus, endovascular coils become permanent medical implants that are commonly used to occlude blood vessels under x-ray guidance for the treatment of multiple different causes of internal bleeding or aneurysm formation using minimally-invasive, non-surgical techniques. In order to facilitate visualization during coil delivery within the blood stream, coils are manufactured using various metals such as platinum, which can be identified using x-rays. However, the presence of a permanent metallic implant such as a coil within the body can create significant artifacts when diagnostic medical imaging tests such as computed tomography (CT) or magnetic resonance imaging (MRI) scans are performed.

These artifacts may render diagnostic medical imaging test interpretation challenging or, in some cases, impossible, which may obscure important or critical findings and negatively influence imaging test performance, thereby harming patient care. Importantly, determining the effectiveness of endovascular coil therapy after treatment of many different causes of internal bleeding or aneurysm formation often requires the use of CT scans or MRIs; it may thus be challenging or impossible in some cases to determine the treatment effectiveness of endovascular coiling when artifact is present. Because a coil is a permanent implant and cannot be removed, these artifacts are permanent throughout the remainder of the patient's life and may continue to negatively impact the interpretation of medical imaging tests under certain circumstances.

To address this, the present invention is directed towards a radiolucent coil—that is, possessing a property of decreased visibility using diagnostic medical imaging tests— with a temporary radiodense outer coating, enabling it to be seen on X-ray images during minimally-invasive delivery within the blood stream. Once this temporary outer coating has dissolved, the coil remains in place within the blood vessel but is of sufficient radiolucency such that it does not generate artifacts on diagnostic medical imaging tests including CT or MRI scans, allowing for improved interpretation. This may also enable better diagnostic medical imaging test performance and earlier identification of important or critical findings that may impact patient care. It may also allow for improved evaluation of treatment effectiveness when coils are used for treatment of multiple different causes of internal bleeding or aneurysm formation.

Thus, the radiolucent endovascular coil of the present invention enables doctors to perform a minimally-invasive, non-surgical treatment of many different causes of internal bleeding or aneurysm formation that does not create artifacts on diagnostic medical imaging tests such as CT or MRI scans. This allows for improved interpretation of these medical imaging tests, which may positively impact patient care. The scale of this issue is very large; both coil placement and diagnostic medical imaging tests are performed daily in health-care facilities throughout the entire world, and endovascular coils are used to treat many different causes of internal bleeding and aneurysm formation in organs and blood vessels throughout the entire body, including the brain.

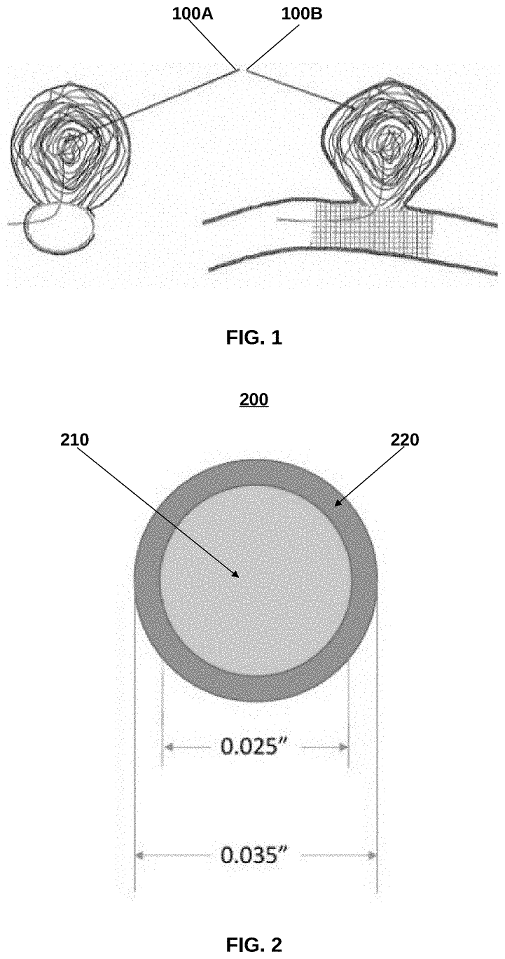

As shown in FIG. 1, coils 100A, 1006 usually form little baskets of different sizes (depending on the size of the coil, which is related to the highest curvature a coil can take). For larger aneurysms or other abnormalities of blood vessels, such as may be associated with internal bleeding, multiple coils may have to be placed, with each coil abutting the previously placed coil(s). Therefore, each aneurysm or abnormal blood vessel requires a careful choice of coils and a sequence of placement. For treatment planning, a physician has to decide which coils should be used and in which order.

In FIG. 2, a cross section of an exemplary coil 200 is illustrated. The exemplary coil 200 includes a radiolucent permanent shape memory (or shape-retaining) polymer core 210 and a temporary degradable radiopaque coating 220. In one embodiment, as shown, the radiolucent permanent shape memory (or shape-retaining) polymer core 210 has a diameter of 0.025 inches, with the temporary degradable radiopaque coating 220, the coil having a diameter of 0.035 inches. Thus, in this embodiment, the temporary degradable radiopaque coating 220 has a thickness of 0.010 inches.

In other embodiments, the radiolucent permanent shape memory (or shape-retaining) polymer core 210 and the temporary degradable radiopaque coating 220 have other diameters. The radiolucent permanent core 210 can be of a shape memory polymer such as polytetrafluoroethylene, polylactide, ethylene vinyl acetate, or the like. A suitable polymer core can also be formed using a more conventional shape-retaining polymer, such as a nylon class that can be set at high temperature (near a glass transition state) to its final form and then straightened for catheter delivery, upon which time the original shape returns with emergence from the catheter. The radiopaque coating 220 may be composed of a material that is broken down and absorbed by hydrolysis, such as, for example, vicryl (a copolymer of lactide and glycoside) or monocryl (a copolymer of glycolide and epsilon-caprolactone). Alternative materials suitable for the degradable coating include natural materials such as, for example, gelatin, chitin, chitosan, and starches. Synthetic materials suitable for the degradable coating include, for example, poly(glycolic acid) and derivatives, and polyphosphazenes. Blending of multiple polymer components is also possible, providing for adjustment of degradation rates, such as incorporating polycaprolactone into a starch preparation.

In embodiments, the temporary degradable radiopaque coating possesses a linear attenuation coefficient equivalent number of at least 700 or more Hounsfield units to provide for adequate contrast for computed tomography imaging and fluoroscopy.

In embodiments, the temporary degradable radiopaque coating is affected by pH and dissolves at physiologic pH of about 7.4.

In embodiments, the temporary degradable radiopaque coating dissolves through interaction with water.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. An endovascular coil comprising:
a radiolucent permanent shape memory or shape-retaining polymer core that retains its coil shape with passage through a catheter; and
a temporary degradable radiopaque coating surrounding the permanent shape memory or shape-retaining polymer core.

2. The endovascular coil of claim 1 wherein a diameter of the radiolucent permanent shape memory or shape-retaining polymer core is in the range of 0.010 to 0.138 inches for applications across the full range of vessel diameters that are suitable for interventional procedures within the brain and elsewhere in the human body.

3. The endovascular coil of claim 2 wherein the thickness of the temporary degradable radiopaque coating is in the range of 0.005 to 0.02 inches.

4. The endovascular coil of claim 3 wherein the temporary degradable radiopaque coating comprises vicryl or monocryl.

5. The endovascular coil of claim 3 wherein the temporary degradable radiopaque coating comprises a natural polymer such as gelatin, chitin, chitosan, and starches, or a synthetic polymer such as poly(glycolic acid) and derivates, and polyphosphazenes, or any suitable degradable biocompatible polymer demonstrating degradation in the desired time frame following placement.

6. The endovascular coil of claim 3 wherein the temporary degradable radiopaque coating comprises a blend of two or more polymer components to provide for adjustment of the degradation rate.

7. The endovascular coil of claim 3 wherein the temporary degradable radiopaque includes iodine in either ionic or covalent form to produce a desired radiopacity.

8. The endovascular coil of claim 3 wherein the temporary degradable radiopaque comprises barium or tantalum.

9. The endovascular coil of claim 1 wherein the temporary degradable radiopaque coating possesses a linear attenuation coefficient equivalent number of 700 or more Hounsfield units to provide for adequate contrast for endovascular placement using x-ray fluoroscopy.

10. The endovascular coil of claim 1 wherein the temporary degradable radiopaque coating is affected by pH and dissolves at physiologic pH of about 7.4.

11. The endovascular coil of claim 1 wherein the temporary degradable radiopaque coating dissolves through interaction with water.

12. An endovascular coil device comprising:
a catheter; and
an endovascular coil, the endovascular coil comprising: a radiolucent permanent shape memory or shape-retaining polymer core; and
a temporary degradable radiopaque coating surrounding the permanent shape memory or shape-retaining polymer core.

13. The endovascular coil device of claim 12 wherein a diameter of the radiolucent permanent shape memory or shape-retaining polymer core is in the range of 0.010 to 0.138 inches for applications across the full range of vessel diameters that are suitable for interventional procedures.

14. The endovascular coil device of claim 12 wherein the temporary degradable radiopaque coating consists of a material such as vicryl or monocryl.

15. The endovascular coil device of claim 12 wherein the temporary degradable radiopaque coating consists of a material is a natural polymer such as gelatin, chitin, chitosan, and starches, or a synthetic polymer such as poly(glycolic acid) and derivates, and polyphosphazenes, or any suitable degradable biocompatible polymer demonstrating degradation in the desired time frame following placement.

16. The endovascular coil device of claim 12 wherein the temporary degradable radiopaque coating consists of a blend of two or more polymer components to provide for adjustment of the degradation rate.

17. The endovascular coil device of claim 12 wherein the temporary degradable radiopaque coating comprises iodine in either ionic or covalent form to produce a desired radiopacity.

18. The endovascular coil device of claim 12 wherein the temporary degradable radiopaque coating further comprises barium or tantalum.

19. The endovascular coil device of claim 12 wherein the thickness of the temporary degradable radiopaque coating is in the range of 0.005 to 0.02 inches.

20. The endovascular coil device of claim 12 wherein the temporary degradable radiopaque coating dissolves through interaction with water.

21. The endovascular coil device of claim 12 wherein the temporary degradable radiopaque coating is affected by pH and dissolves at physiologic pH of about 7.4.
